(19) United States
(12) Patent Application Publication
(10) Pub. No.: US 2012/0298114 A1
(43) Pub. Date: Nov. 29, 2012

(54) WOUND DRESSING SYSTEM

(75) Inventors: Kelly Landsman, Milwaukee, WI (US); Shayna Massi, Chicago, IL (US)

(73) Assignee: CareFusion 2200, Inc., San Diego, CA (US)

(21) Appl. No.: 13/116,826

(22) Filed: May 26, 2011

(51) Int. Cl. A61B 19/08; A61F 13/56; A61M 27/00

(52) U.S. Cl. 128/849; 604/543; 604/385.03

(57) ABSTRACT

System, method, and components for hygienic maintenance of a surgical wound configured for drainage of ascites from a body cavity. Different aspects of the system may include one or more of a wound dressing, a pocket for low-profile, hygienic, and aesthetic storage of a drainage catheter configured for attachment to a patient and/or wound dressing, and an apron device configured for installation and/or exchange of a wound dressing.

### TECHNICAL FIELD

Embodiments of the present invention relate to the field of removing peritoneal ascites, pleural effusion fluids, and the like. More particularly, embodiments of the present invention relate to a wound dressing system configured for infection prevention at the site of a fluid drainage exit site of a patient.

### BACKGROUND

Ascites describes an accumulation of fluid in the peritoneal cavity. Pleural effusion refers to the effusion of fluid into the pleural space. Both excess fluid accumulation conditions may be treated with a drainage apparatus of the type shown in FIG. 1. The apparatus 100 is shown as installed in a patient body and includes a drainage container 114. The drainage container 114 is removably attached by a proximal drainage line 110 at a valve 60 to a distal catheter 12. The valve 60 may be configured in any number of ways known in the art for attaching catheters together in a fluid-patent manner using a single valve or a dual component valve. For example, the valve may be configured in the manner described in U.S. Pat. No. 5,484,401 to Rodriguez, et al., which is incorporated by reference herein. The proximal portion attached to the distal catheter 12 may be configured to be self-sealing when disconnected from the proximal drainage line 110. The proximal end portion of the proximal catheter 12 is shown indwelling the patient, disposed through the body wall 21 into an intra-body space 23, which may be—for example—a pleural, peritoneal, or other body cavity. That proximal portion includes a sealing cuff 19 and a flexible fluid-intake length 14 including apertures 18, shown in the intra-body space 23. This structure may be better understood with reference to U.S. Pat. No. 5,484,401, which is incorporated herein by reference, and with reference to commercial products marketed under the name PleurX by CareFusion® of San Diego, Calif.

The pleural space normally contains approximately 5 to 20 ml of fluid. The pH, glucose and electrolytes of the fluid are equilibrated with plasma, but the fluid is relatively protein-free. The fluid is the result of the hydrostatic-oncotic pressure of the capillaries of the parietal pleura. About 80-90% of the fluid is reabsorbed by the pulmonary venous capillaries of the visceral pleura, and the remaining 10-20% is reabsorbed by the pleural lymphatic system. The turnover of fluid in the pleural space is normally quite rapid—roughly 35 to 75% per hour, so that 5 to 10 liters of fluid move through the pleural space each day.

A disruption in the balance between the movement of fluid into the pleural space and the movement of fluid out of the pleural space may produce excessive fluid accumulation in the pleural space. Such disruptions may include, for example, (1) increased capillary permeability resulting from inflammatory processes such as pneumonia, (2) increased hydrostatic pressure as in congestive heart failure, (3) increased negative intrapleural pressure as seen in atelectasis (partial or total lung collapse), (4) decreased oncotic pressure as occurs in the nephrotic syndrome with hypoalbuminemia, and (5) increased oncotic pressure of pleural fluid as occurs in the inflammation of pleural tumor growth or infection. Pleural effusion is particularly common in patients with disseminated breast cancer, lung cancer or lymphatic cancer and patients with congestive heart failure, but also occurs in patients with nearly all other forms of malignancy.

The clinical manifestations of pleural effusion include dyspnea, cough and chest pain which diminish the patient's quality of life. Although pleural effusion typically occurs toward the end of terminal malignancies such as breast cancer, it occurs earlier in other diseases. Therefore relieving the clinical manifestations of pleural effusion is of a real and extended advantage to the patient. For example, non-breast cancer patients with pleural effusion have been known to survive for years.

There are a number of treatments for pleural effusion. If the patient is asymptomatic and the effusion is known to be malignant or paramalignant, treatment may not be required. Such patients may develop progressive pleural effusions that eventually do produce symptoms requiring treatment, but some will reach a stage where the effusions and reabsorption reach an equilibrium that is still asymptomatic and does not necessitate treatment.

Pleurectomy and pleural abrasion is generally effective in obliterating the pleural space and, thus, controlling the malignant pleural effusion. This procedure is done in many patients who undergo thoracotomy for an undiagnosed pleural effusion and are found to have malignancy, since this would prevent the subsequent development of a symptomatic pleural effusion. However, pleurectomy is a major surgical procedure associated with substantial morbidity and some mortality. Therefore, this procedure is usually reserved for patients with an expected survival of at least several months, who are in relative good condition, who have a trapped lung, or who have failed a sclerosing agent procedure.

In general, systemic chemotherapy is disappointing for the control of malignant pleural effusions. However, patients with lymphoma, breast cancer, or small cell carcinoma of the lung may obtain an excellent response to chemotherapy. Another approach to removing fluid from the pleural space has been to insert a chest tube. Such tubes are commonly quite rigid and fairly large in diameter and are inserted by making an incision and spreading apart adjacent ribs to fit the tube into place. Such procedures are painful to the patient, both initially when the chest tube is inserted and during the time it remains within the pleural space.

Thoracentesis is a common approach to removing pleural fluid, in which a needled catheter is introduced into the pleural space through an incision in the chest cavity and fluid is positively drawn out through the catheter using a syringe or a vacuum source. The procedure may also include aspiration utilizing a separate syringe. There are a number of difficulties in thoracentesis, including the risk of puncturing a lung with the catheter tip or with the needle used to introduce the catheter, the risk of collapsing a lung by relieving the negative pressure in the pleural space, the possibility of aggravating the pleural effusion by stimulating fluid production in the introduction of the catheter/drainage line, and the risk of infection. One of the primary difficulties with ordinary thoracentesis procedures is that fluid reaccumulates in the pleural space relatively quickly after the procedure is performed, and so it is necessary to perform the procedure repeatedly—as often as every few days.

Do we need the same level of detailed background for ascites treatment modalities?

Modern pleural and peritoneal drainage systems have made it possible for patients to use devices like those illustrated in FIG. 1 to conduct drainage in the home setting, either on their own or with the help of a caregiver. However, the current standard dressings often require removal to provide access to the proximal valve portion 60, which exposes the catheter/drainage line exit site from the patient's body wall. This may present increased risk of certain types of infection, particularly if a patient desires to conduct a drainage therapy at home, outside the more controlled environs often available in a hospital or a physician's office. Some dressing types may allow a drainage session without exposing the catheter exit site, but the external catheter portion is then often just taped to the patient's body, where the valve end 60 may remain exposed to leak or allow invasion of infectious agents that could travel into the patient.

Accordingly it may be desirable to provide a wound dressing system that provides for a patient's ability to conduct a drainage session at home without exposing his/her surgical wound/port. It may also be desirable to provide a wound dressing system that also provides clean safe storage of the distal catheter portion that is external to the patient's body.

### BRIEF SUMMARY

In one aspect, embodiments of the present invention may include a wound dressing system and installation apron for same, as well as methods for installation. In other aspects, embodiments of wound dressing systems may include a pocket for storing an external length of drainage catheter in a generally clean, convenient, low-profile, and aesthetically acceptable manner.

### BRIEF DESCRIPTION OF THE DRAWINGS

FIG. 1 shows a drainage apparatus of the type used for drainage of pleural or peritoneal ascites;

FIGS. 2A-2C show an embodiment of a pocket device;

FIGS. 2D-2E show another embodiment of a pocket device with a cap;

FIG. 2F shows a pocket device embodiment as a component of a wound dressing assembly;

FIGS. 3A-3B show another pocket device embodiment as a component of a wound dressing assembly;

FIG. 4 shows yet another pocket device embodiment as a component of a wound dressing assembly;

FIGS. 5A-5B show still another pocket device embodiment as a component of a wound dressing assembly;

FIG. 6 shows another pocket device embodiment as a component of a wound dressing assembly;

FIG. 7 shows a valve with hygienic maintenance contact indicia;

FIGS. 8A-8D show a wound monitoring system; and

FIGS. 9A-9E show an apron system configured for hygienic wound maintenance.

### DETAILED DESCRIPTION

Embodiments are generally described with reference to the drawings in which like elements are generally referred to by like numerals. The relationship and functioning of the various elements of the embodiments may better be understood by reference to the following detailed description. However, embodiments are not limited to those illustrated in the drawings. It should be understood that the drawings are not necessarily to scale, and in certain instances details may have been omitted that are not necessary for an understanding of embodiments of the present invention, such as—for example—conventional fabrication and assembly.

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise. Throughout the specification, the terms “distal” and “distally” shall denote a position, direction, or orientation that is generally away from the physician and/or toward the patient. Accordingly, the terms “proximal” and “proximally” shall denote a position, direction, or orientation that is generally towards the physician and/or toward the patient.

Presently, typical exit site dressings for pleural drainage, peritoneal drainage, and other similar ports/apertures in a patient's skin include an adhesive patch that secures to the patient's skin a catheter that extends through the surgical wound. The external catheter portion (including its proximal end having the valve) often is secured under the same dressing. Alternatively, it may be separately taped or otherwise secured to the patient's body. Various cap mechanisms are known in the art that may be secured to the proximal valve end. These and other means are generally configured to provide for drainage and to prevent bacteria or other infectious agents from entering the body via the catheter. However, for the patient, this arrangement can pose several challenges with regard to physical comfort, as well as psychological well-being connected with self-image.

When the proximal end is taped or otherwise secured to the patient's skin, it may be irritating or painful to remove that portion to allow drainage through the catheter, without dislodging/disturbing the primary exit site wound dressing. The patient's skin may become irritated from the removal and reapplication of adhesives around the proximal catheter end. However, it is often impractical (and potentially unsafe) to leave the proximal catheter end unsecured, as it may catch on the patient's clothing, it may be visually/aesthetically displeasing to the patient (and/or to others, with a negative impact on patient psyche), and/or it may leak liquids and/or gases from the drainage site at the distal catheter portion. In addition, if not properly cared for, the exit site dressing and/or the loose/external catheter end may allow infectious agents to enter a patient's body.

In one aspect, certain embodiments described herein provide a wound dressing system including an adhesive patch configured to adhere to a patient body site around a catheter exit site, wherein the adhesive patch includes an aperture configured for passage therethrough of a catheter disposed through the catheter exit site, a sealing structure configured to seal between the catheter and the patch to form a barrier to the catheter exit site, a low-profile pocket configured for attachment to the patch or as another layer of the patch, and configured to substantially contain and conceal a proximal catheter length within the low-profile pocket. One unifying concept of embodiments described herein may include a goal of maintaining sterile or at least hygienically-desirable conditions for a drainage catheter. In particular, it may be desirable to promote these conditions in a manner easily applicable by a patient and/or attending personnel who are not trained medical caregivers (i.e., persons other than physicians, nurses, or the like).

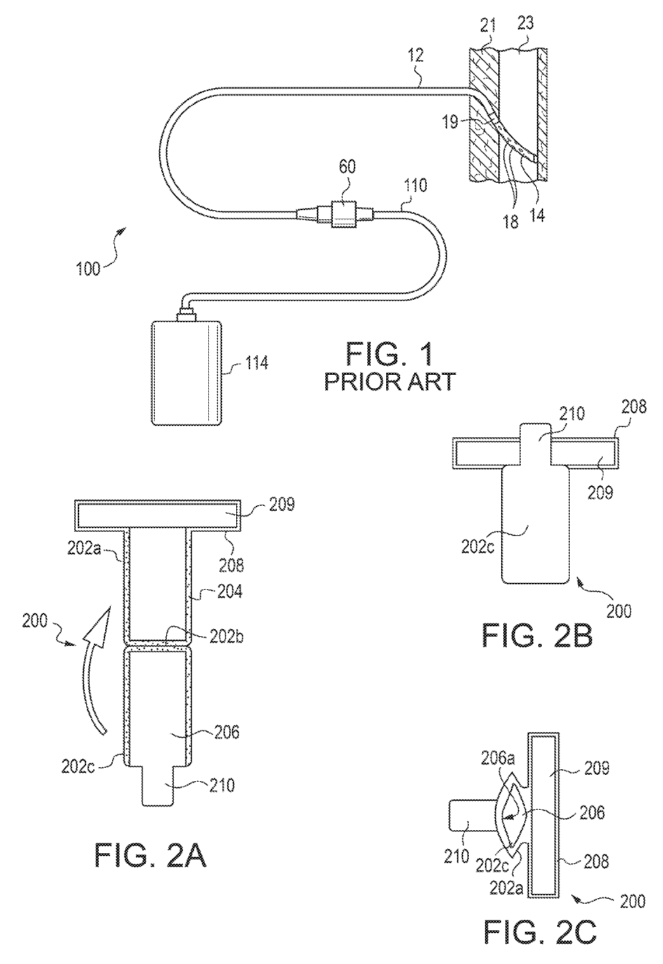

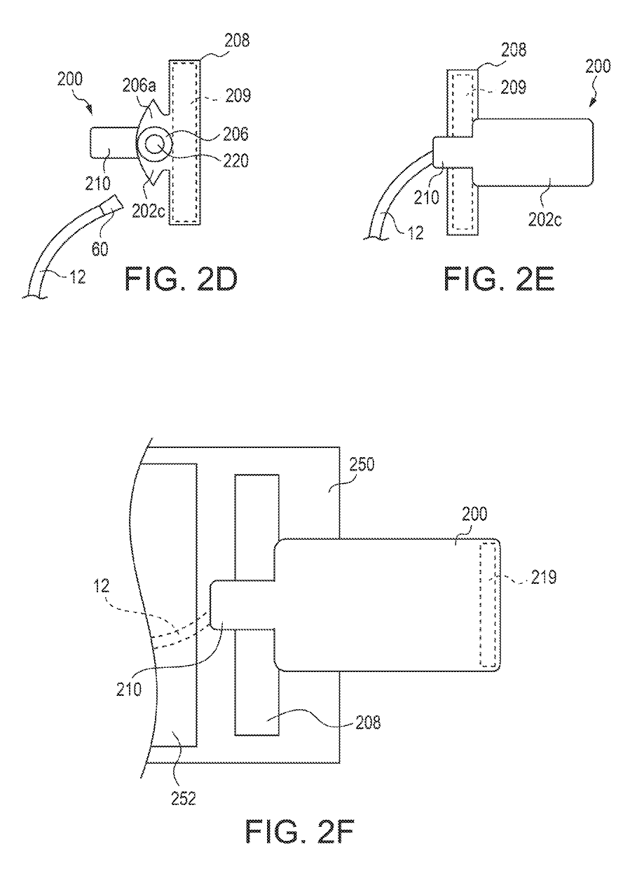

FIGS. 2A-2C show one embodiment of a pocket 200, configured to contain and conceal an external proximal catheter length as part of a wound dressing system. The pocket 200 may thus protect the catheter and the patient while also providing low-profile storage for the catheter. In addition to decreasing the likelihood of the catheter moving in an undesired manner, the lower profile pocket may provide a dressing system that presents reduced aesthetic concern to patients. It may also present advantages for infection prevention by protecting the catheter exit site, catheter, and valve (e.g., during showering, daily wear, etc.).

In contrast with existing systems that adhere the external catheter portion to a patient's body, the pocket 200 of the present embodiments may provide ease of access without the discomfort associated with removing an adhesive patch from the patient's skin (holding the catheter thereto) in the manner of said existing systems. FIG. 2A shows the pocket in a “disassembled” configuration. A first pocket portion 202a is configured as attached or an integral additional part of a second pocket portion 202c along a fold-line 202b. Three edges of the first and second pocket portions 202a, 202c are configured as an attachment region 204 to be attached together around—and thereby to form—a central pocket compartment 206. The attachment may be formed by—for example—adhesive, ultrasonic welding, or any other appropriate connection means. Alternatively, the pouch may be seamlessly molded to form the central pocket compartment. In certain preferred embodiments, the central pocket compartment may include an absorbent and/or cushioning member, and/or be constructed with an absorbent internal surface 206a and leak-resistant external surface, configured to prevent the valve from contamination during bathing or external sources, absorb and provide cushioning. Exemplary absorbent materials may include foam, cotton, non-wovens, and exemplary leak-resistant materials may include plastic films.

An end of the first pocket portion 202a opposite the fold line 202b includes a laterally-extending strip 208. The strip 208 may have an attachment region 209 including on the underside at least one of a skin-safe adhesive, an adhesive configured for securing it to a wound dressing, or other securing means configured to permanently or removably attach the pouch 200 to the patient and/or wound dressing (e.g., hook-and-loop fasteners such as Velcro™, tongue-and-groove fasteners like zip-lock, or any other appropriate mechanical fastening or securing structure). An end of the second pocket portion 202c opposite the fold line may include a longitudinally-extending tab 210, which may be configured to tuck into the central pocket compartment 206, or may remain extended. If folded in, this tab 210 may help to secure and conceal the proximal catheter portion substantially within the central pocket compartment 206. The tab 210 may have adhesive which allows it to seal around the catheter entering the pouch.

FIG. 2B shows a plan view of the pouch 200, assembled. FIG. 2C shows a top view of the pouch 200, with the central pocket compartment 206 held open and the tab 210 and strip 208 each held back for visual clarity. FIG. 2D shows a top view of different embodiment of the pouch 200, with the central pocket compartment 206 held open. A cap 220 is shown in the compartment 206, ready to receive/engage the valve portion 60 of a catheter length 12 external to a patient. The cap 220, which may be configured as a valve cover constructed to shield or seal a distal valve end, may be attached to or integrated with the pouch 200. FIG. 2E shows the catheter 12 being tucked into the central pocket compartment 206.

FIG. 2F shows the pouch 200 assembled as part of a wound dressing assembly. A base portion 250 of the wound dressing patch is secured to the patient's skin around a catheter exit site (not shown). A sealing cover structure 252 configured to seal between the catheter 12 and/or around the patch to form a barrier to the surgical wound. Substantially the entire portion of the catheter 12 external to the patient and the sealing cover structure 252 is tucked/coiled into the compartment 206. The pouch strip 208 is attached to or part of the patch 250. The side of the pouch 200 adjacent the patient's skin may be configured to include a region of skin-safe adhesive 219 configured to keep the pouch 200 close to the patient's body exterior. In preferred embodiments, the pouch is constructed to provide a low profile that will not extend far from the body and that will keep the pouch and any contents close enough that they may be minimally visible or substantially imperceptible under a patient's clothing. In this embodiment, the adhesive 209 on the strip portion 208 is opposite the tab. However, in other embodiments, it may be on the same side, such that the catheter will be disposed between the adhesive 209 and the tab 210.

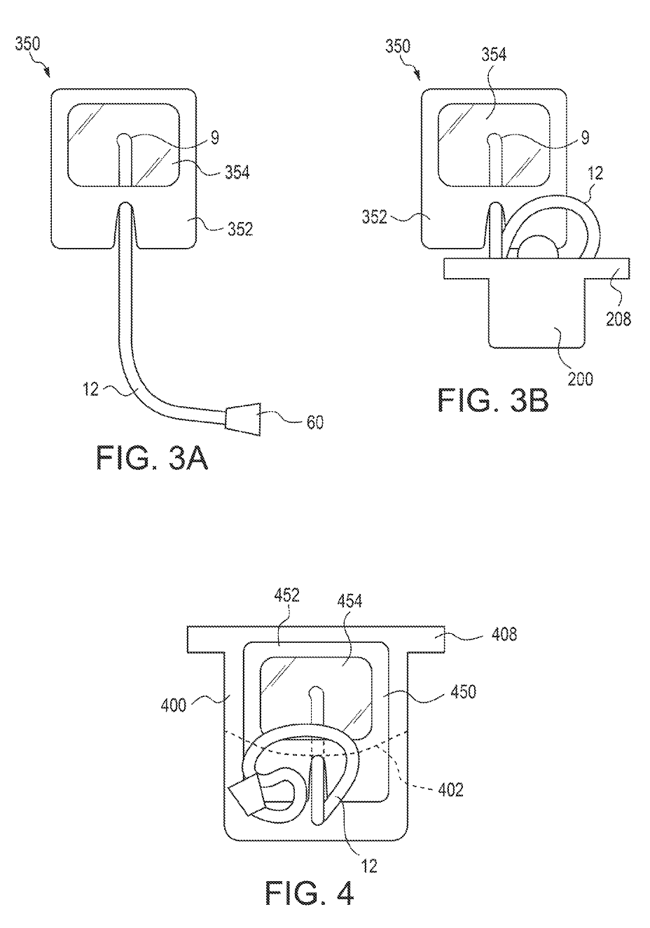

FIGS. 3A-3B show a system using an embodiment of the pouch 200. A windowed patch 350 is provided including an adhesive border 352 and a window 354 that preferably is substantially transparent, and that may also include adhesive. The patch 350 anchors the catheter 12 into a catheter exit site 9. The adhesive border 352 seals to and around the catheter 12 where the catheter 12 including a proximal valve end 60 extends out from underneath the patch 350. The pouch 200 may be mounted by its strip 208 to the patch 350 and/or to a patient's skin adjacent the patch 350. As shown in FIG. 3B, the external portion of the catheter 12 that extends out from under the patch 350 may be coiled and stored in the low-profile pouch 200. The window 354, when transparent, will allow the patient and/or attending personnel to monitor the condition of the exit site 9.

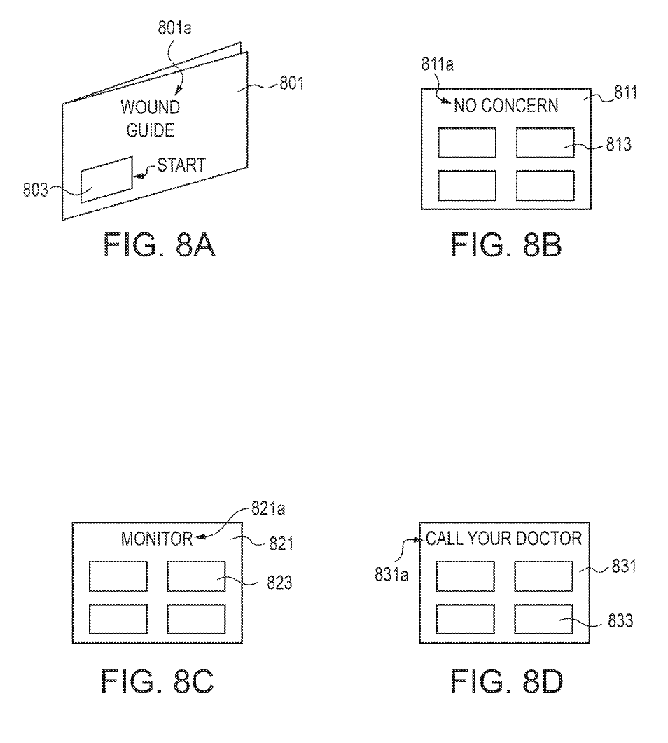

This wound-monitoring feature of the window 352 may be utilized in a system further incorporating a wound-monitoring system described with reference to FIGS. 8A-8D. Each of FIGS. 8A-8D includes a card with reference photographs (shown here in blank/diagrammatic form only) for a patient and/or attending personnel to use in monitoring the wound 9. FIG. 8A is a primary reference card 801 with a photograph 803 intended to show the wound, with or without a catheter in place, in its beginning state (i.e., a photograph taken soon after the opening is made). FIG. 8B shows a second reference card 811 with at least one or more images 813 (e.g., photographs and/or diagrams)—preferably in color—of healthy wound/port sites not requiring any special treatment, that not requiring any change in the state of the wound and dressing. FIG. 8C shows a third reference card 821 with at least one or more images 823—preferably in color—of wound sites that are less than healthy (e.g., showing early signs of infection, necrosis, inflammation, or other unhealthy conditions) and that should be monitored closely for any negative changes. FIG. 8D shows a fourth reference card 831 with at least one or more images 833—preferably in color—of wound sites that are clearly in an unhealthy state, and that should be viewed and potentially treated by a health care professional. Stated differently, FIG. 8C may be characterized as including at least one reference image showing a need for closer monitoring, and FIG. 8D may be characterized as including at least one reference image showing a state of healthiness of the site that indicates a need for intervention (e.g., change of the dressing, treatment by a health care professional, or other change).

As shown in FIGS. 8A-8D, each of the sets of diagrams/photos may be labeled with a legend (such as, for example, 801a, 811a, 821a, 831a) for easy reference by the patient. In this manner, even an untrained patient, caregiver, or other person may monitor the wound site by periodic comparison of its appearance through the window 302 with the photos/diagrams as in FIGS. 8A-8D. Those conditions may be logged (e.g., by photographs, narrative in a diary or other record, etc.). In the event that the wound is not healthy, the patient or other attending personnel will know what appearance merits closer monitoring versus a need to call a physician. The cards and/or reference images may be separate, or may all be mounted together (e.g., on a wall, in a book, in a portable pocket-sized packet, or any other convenient form). The cards 801, 811, 821, 831 may be mounted on a single surface, compiled into a “flip-book,” assembled together in some other manner, or stored separately. The images may be compiled in a compact form (e.g., fold-out on thin durable paper) that could be attached to a wound dressing or pocket of the embodiments described. Alternatively, or in addition, the images may be saved in digital format (e.g., on a compact memory card such as a microSD, CF, USB memory stick/thumb drive or the like), which may provide for ready viewing on a computer, camera, or mobile device such as—for example a smartphone, media player, or other device.

FIG. 4 shows another embodiment of a wound dressing system including an oversized pouch 400. But for its relative size, the pouch 400 may be constructed like that shown in FIGS. 2A-2C, except with a lower pocket/compartment, the upper edge of which is indicated by dashed line 402. For the sake of illustrative clarity, the wound dressing structure under the pouch 400, including a patch 450 with a window 454 surrounded by an adhesive border 452 and coiled catheter 12 extending from beneath the patch into the pocket, is shown in solid lines except for the upper edge of the pocket's compartment. However, in this embodiment, the dressing is disposed beneath and covered by the pouch 400. That is, the wound dressing (450 et al.) is between the pouch 400 and the patient's skin. The pouch 400 includes a strip portion 208 for mounting the patch to the underlying dressing and/or patient skin, where it can function as a hinge, allowing the pouch to be lifted and the catheter 12 tucked into its inner compartment/pocket as shown in FIG. 4.

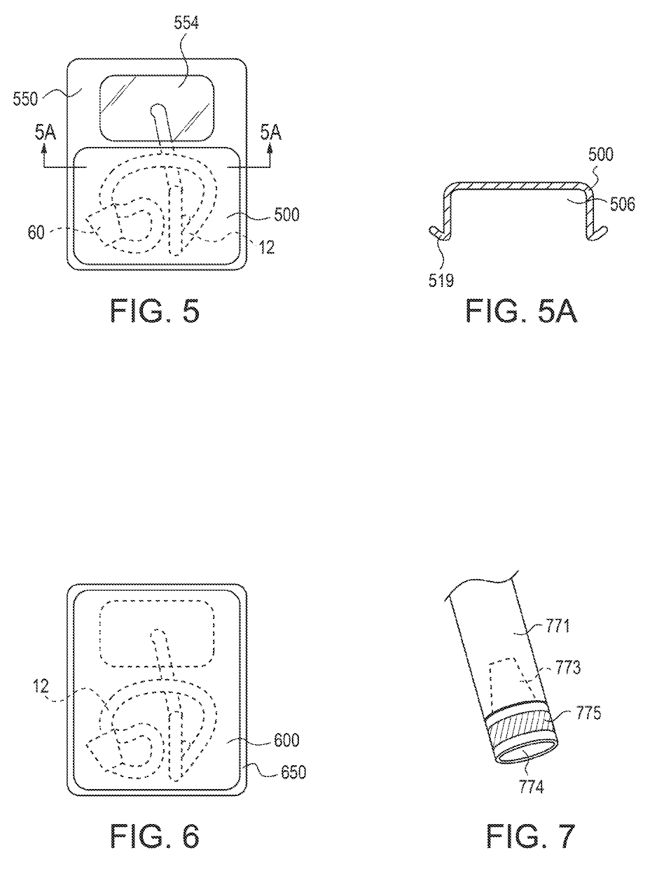

FIG. 5 shows another embodiment of a wound dressing system. As described with reference to FIGS. 5 and 5A, a patch 550 is provided with a substantially transparent window 554, through which a catheter's (12) entry into a patient body is viewable. The patch could have adhesive only in the window and around the outside border to maintain a fluid-tight seal while reducing adhesive irritation. The patch could be made of material layers that are fluid tight and cushioning. The patch 550 also includes an opening (not shown) configured to allow transit therethrough of the catheter 12. This feature of wound patches is well-known in the art and may be embodied in any manner known in the art or yet developed while being practiced within the present invention. A pouch member 500 is also provided, and is configured to attach completely to the patch 550, such that a pouch compartment 506 is formed therebetween. The pouch member 500 and patch 550 are configured to engage together, preferably in a manner forming a seal that preferably is at least substantially liquid-tight, such that it will have a low risk of allowing the catheter 12 to be contaminated from water during showering. In this embodiment, the pouch member 500 is dimensioned, and oriented to attach only to a portion of the patch 550 that will not obscure the window 554, which will allow the patient to monitor his/her wound condition without exposing the external portion of the catheter 12, or a valve portion 60 thereof (if present).

FIG. 5A shows a lateral section view of the pouch member 500 along line 5A-5A of FIG. 5. A lower connection portion 519 of the pouch member 500 may be configured in a variety of ways to attach (including to sealingly attach) to the patch 550. For example, the patch 550 and pouch 500 may be constructed with complementary interlocking tongue-and-groove structures (e.g., zip-lock and/or snap-fit style configuration). As another example, one or both of the contacting surfaces may include an adhesive material. As yet another example, a hook-and-loop (e.g., Velcro™-type) interface may be used to form a connection. Flexible magnetic strips or any other appropriate mechanical or chemical means for attachment may be used.

Those having skill in the art will appreciate in view of the present disclosure that a variety of connecting, fastening, and/or securing means may be implemented to attach the pouch member 500 to the patch 550, leaving a space/compartment 506 to house a portion of the catheter 12, which may include a valve portion 60, external to the patient body and the patch 550. The pouch member 500 preferably will be at least semi-flexible to promote patient comfort and maintain a low profile relative to the patient body and the patch 550. The border 519 of the pouch member 500 may be somewhat less flexible than the rest of it, if needed to form and maintain a connection (up to and including a liquid-tight sealing connection) with the patch 550.

FIG. 6 shows an embodiment of a wound dressing system similar to that shown in FIG. 5, including a patch 650 and a pouch member 600. Like the embodiment of FIG. 5, the pouch member 600 is attached around its edges to the patch 650. However, in this embodiment, the pouch member 600 is configured to cover all or substantially all of the patch 650, which may include a window or just an open aperture configured to permit transit of a catheter length 12. Like the embodiment described with reference to FIGS. 5-5A, the attachment means may be configured to form a liquid-tight seal and may be configured in any appropriate manner for attachment and removability.

FIG. 7 shows another embodiment of a portion of a wound dressing system. The drawing shown provides representation applicable to one or both of a proximal or distal ends of a catheter 771, including a valve 773. In order to promote hygienic handling of components of a patient care system, it is often preferable only to touch portions of the system that will not be contacting elements or materials that will enter a patient's body or that could otherwise cause contamination. As such, a portion of the valve 773 is marked with indicia 775 to show a patient or other caregiver where it is appropriate to touch/grasp the valve 773. The indicia 775 may include one or more of coloration, physical texturing, wording, or other visual and/or tactile indicia. In another aspect, contrasting indicia may be used for other portions of the system. For example, the portions that are intended for being touched/grasped may be color-coded green, while the other portions (adjacent or otherwise) that preferably should not be touched may be color-coded red. For example, it may be desirable that no one should touch the interior surface 774 of the valve 773, and that portion may be coded appropriately with some desired indicia.

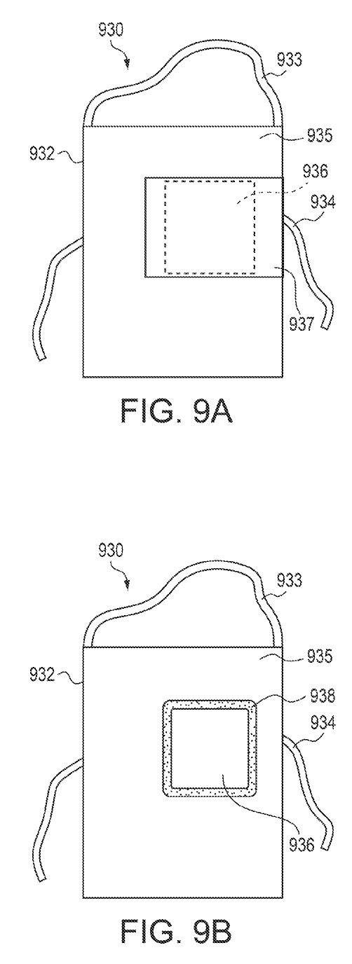

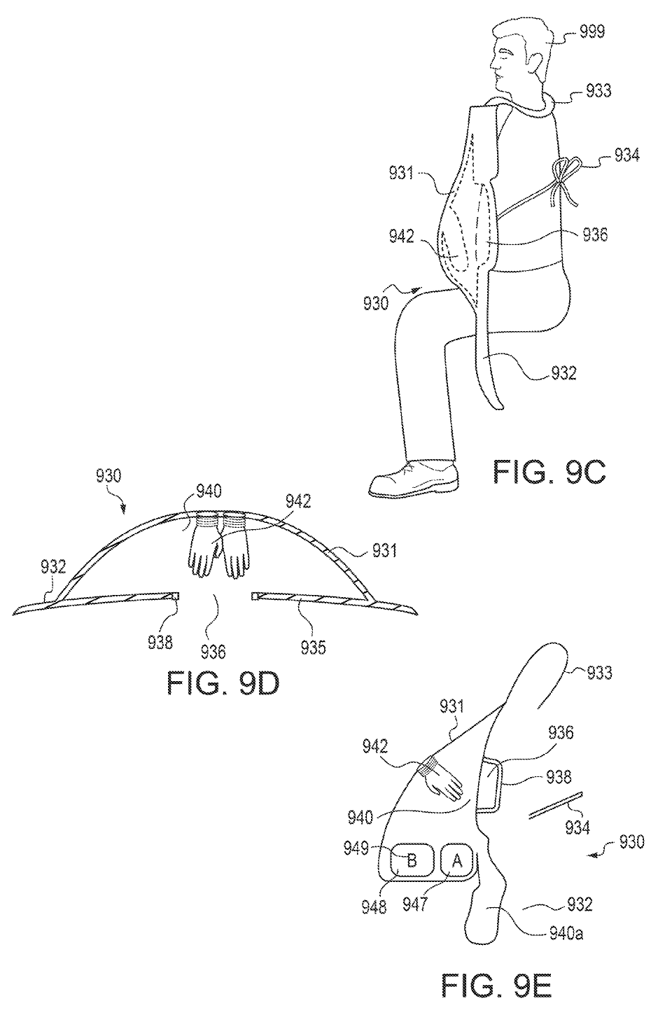

An embodiment of an apron system 930, configured as an extension of a wound dressing system for hygienic installation thereof, is described with reference to FIGS. 9A-9E, each of which is portrayed diagrammatically to show the internal components of the system without incorporating complicating visual details showing specific cuts of section lines. In view of the following disclosure, this diagrammatic approach will readily be understood by those of skill in the art. FIG. 9A shows a rear (patient-side-facing) view of the system 930, which includes an apron member 932. The apron member 932 may include a neck loop 933 and tie straps 934 to help secure it to a patient. The rear wall 935 of the apron member 932 includes an aperture 936, that is shown as covered by a removable cover 937 in FIG. 9A and open (uncovered) in FIG. 9B. The aperture 936 is open to provide communication with a compartment 940 enclosed by the apron member 932. The border 938 of the aperture 936 may be configured to be attached to a surface (e.g., a patient body region around a wound to be treated). The border 938 may include a skin-safe adhesive or other means known in the art that preferably is configured to maintain a secure but removable hygienic seal around the aperture 936.

FIG. 9C shows an embodiment of the apron member 932 installed on a patient 999. The apron 932 is shown diagrammatically in partial longitudinal section, such that the interior portion of the compartment 940 is visible, with a side view shown in FIG. 9C and a top lateral section view shown in FIG. 9D. It is secured to the patient 999 around the aperture 936, the border of which is sealed to the patient's skin, as well as by the neck loop 933 and tie straps 934. The compartment 940 is constructed as a hygienically enclosed space that may be sterilizable. A front wall 931 of the apron 932 includes at least one integrated glove (or mitten) member 942, which preferably is configured and oriented to allow dexterous manual manipulation by the patient 999 or a caregiver of an item within the compartment 940. Although gloves are shown and described herein, it should be appreciated that some embodiments may be provided with a mitten-type configuration not requiring separate articulated fingers; such a configuration may provide an advantage for patients having limited fine motor dexterity.

The at least one glove 942 may include at least two gloves and may include four or more gloves to allow multiple users working access to the compartment and/or to provide for “clean use” and “dirty use” gloves. For example, in an embodiment providing two or more pairs of gloves 942 in the front wall 931, a first pair of “dirty use” gloves may be used to remove an existing wound dressing, wrap it up, and clean the surface area. A second pair of “clean use” gloves may be configured and/or used for handling only components for installation of a new, clean wound dressing (such as, for example, one or more components of a wound dressing system embodiment described herein with reference to FIGS. 2A-8D). So that the “clean use” gloves remain uncontaminated or at least have a reduced risk of contact with an old dressing being removed, they may be kept everted (inside out) during usage of the first pair of gloves to remove an old dressing. The gloves may include stretchable and/or telescoping/accordioning sleeves 941 configured to allow the gloves to be manipulated substantially anywhere in the compartment 940 without significantly having to distort the front wall 931. The front wall 931 may be constructed entirely, or at least in part, of a substantially transparent material that will allow visualization of the compartment 940 and aperture 936.

A method of using the apron system 930 is described with reference to FIG. 9E. In the embodiment shown in FIG. 9E, the compartment 940 may be provided as sterile, or nearly sterile, and have enclosed therein at least one medical supply item, such as—for example—some or all components needed to hygienically change a patient's wound dressing. In the embodiment illustrated, the apron member 932 may be mounted onto a patient (not shown) in the manner previously described (e.g., with reference to the seated patient shown in FIG. 9A). The aperture border 938 may be sealed to the patient's body, and the patient or a caregiver may insert his hands into the gloves 942.

In the embodiment of FIG. 9E, two packets of components are provided. A removal packet 947 may include all supplies needed to remove an existing wound dressing, as well as to clean the wound and surrounding skin. For example the removal packet 947 may include alcohol wipes useful for assisting in release of adhesive of an already-installed wound dressing from a patient's skin, cleansing swabs preloaded with a cleaning solution to clean the wound area after removal of an old dressing, and gauze pads to dry the skin area. A compartment portion 940a configured to receive materials in a manner isolating them from the rest of the compartment may be provided for disposal of removed materials and used components. An installation packet 948 may be provided including a replacement wound dressing (e.g., according to one of the embodiments described herein). The packets may be sealed packets of the type known in the art for use with surgical kits, components, and the like, and may include visual or other indicia 949 (e.g., the letters “A” and “B” as shown, and/or any other visual or tactile indicia useful to identify—for example—what a given packet is, and in which order it is intended to be used). After the old wound dressing is removed and disposed of in the disposal compartment portion 940a, the installation packet may be opened and the new dressing installed.

If two pairs of gloves are provided, a first set of gloves may be used for the removal, cleaning, and disposal steps, then everted so the surfaces that had contacted the old dressing no longer extend into the compartment. Thereafter, the patient or caregiver may put his hands into the second pair of gloves, which may previously have been everted to prevent contact with the old dressing and cleaning materials, and which may be used during installation of a new wound dressing. In order to maintain open/unobstructed working space within the compartment 940, the apron 932 may be provided with one or more of supportive struts (e.g., integrated into the apron construction or attached/attachable thereto), an integrated, internal, or external support frame, a generally air-tight construction that may be inflated to greater than ambient pressure during use, and/or another appropriate technological solution that will be appreciated by those having skill in the art. For example, it is contemplated that, in the embodiment shown in FIG. 9E, one or more struts (not shown) or other support structure may be integrated into the apron member's front wall 931, enabling it to maintain the illustrated configuration of being curved or otherwise held out away from the rear wall 935 to maintain sufficiently open space in the compartment 940 to effect a desired task.

In some embodiments, a drainage container and other external components for conducting pleural or peritoneal drainage may be included in the compartment with—or instead of—a replacement wound dressing. For example, a drainage container and catheter having a valve portion complementary to a valve portion on the proximal end of a patient-indwelling catheter may be provided in the apron compartment. Those having skill in the art will appreciate that such a structure and method may help to provide for a hygienic drainage session or at least drainage container installation. For example, a cap on the patient's catheter valve portion may be cleaned and removed only after the apron was secured to the patient, after which the drainage container's catheter could be attached via its valve portion. The apron may be removed with the container attached, or after drainage was completed and the patient's catheter valve portion has been re-capped.

In FIG. 9C, it is contemplated that a patient may be seated generally upright with the major portion of the compartment 940 supported by a table or tray (not shown), while the disposal portion 940a of the compartment 940 is kept separate (e.g., by being allowed to hang between the patient body and the supported compartment portion). Those of skill in the art will appreciate that the apron system described here may be useful for a variety of treatments beyond the wound dressing changes including, for example, other procedures requiring hygienic conditions but not necessarily the level of sterility expected in a hospital or other dedicated care setting).

Those of skill in the art will appreciate that embodiments not expressly illustrated herein may be practiced within the scope of the present invention, including that features described herein for different embodiments may be combined with each other and/or with currently-known or future-developed technologies while remaining within the scope of the claims presented here. Although specific terms are employed herein, they are used in a generic and descriptive sense only and not for purposes of limitation. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment of the invention.

We claim:

1. A wound dressing for a surgical wound configured as a drainage access port for a catheter, the wound dressing comprising:
an adhesive patch configured to adhere to a patient body site around a surgical wound, wherein the adhesive patch includes an aperture configured for passage therethrough of a catheter disposed through the surgical wound;
a sealing structure configured to seal between a catheter and the patch to form a barrier to the surgical wound; and
a low-profile pocket member configured for attachment to a location selected from the patch, the patient body site, and a combination thereof,
wherein the low-profile pocket member includes a pocket member compartment configured to substantially contain and conceal a catheter length.

2. The wound dressing of claim 1, wherein the pocket member comprises absorbent material.

3. The wound dressing of claim 1, wherein the pocket member's configuration for attachment includes a strip that includes at least one of adhesive or a mechanical fastening structure.

4. The wound dressing of claim 1, wherein the pocket member is configured to cover substantially the entire patch.

5. The wound dressing of claim 1, further comprising a catheter member attached to the patch and having an end portion disposed in the pocket member pouch.

6. The wound dressing of claim 1, wherein the pocket member compartment comprises a cap configured to cover a catheter end.

7. The wound dressing of claim 1, wherein the pocket member comprises a cap configured to cover a valve portion disposed upon a catheter end.

8. The wound dressing of claim 1, wherein the patch comprises a substantially transparent window configured to allow viewing of a patient surface region below the window.

9. The wound dressing of claim 8, further comprising a wound monitoring guide comprising at least one first reference image of a patient surface region in a first state of healthiness, where the first state is a state not requiring any change in the wound dressing on a patient surface region.

10. The wound dressing system of claim 9, further comprising at least one second reference image in a second state of healthiness, where the second state of healthiness indicates a need for intervention.

11. The wound dressing system of claim 9, further comprising at least one second reference image in a second state of healthiness, where the second state of healthiness indicates a need for closer monitoring.

12. The wound dressing system of claim 1, wherein a border of the pocket member and a surface of the patch are configured to engage together.

13. The wound dressing system of claim 12, wherein substantially an entire border encompassing the pocket member engages at least a portion of the patch, and wherein the compartment comprises a space between the pocket member and the patch.

14. The wound dressing system of claim 13, wherein an engagement of the pocket member with the patch comprises a substantially liquid-tight seal.

15. The wound dressing system of claim 13, wherein the compartment is configured and dimensioned to accommodate a length of catheter extending from beneath the patch.

16. The wound dressing of claim 1, further comprising a catheter at least partially disposed beneath the patch, wherein the catheter comprises an exposed proximal portion, and wherein the exposed proximal portion comprises indicia designating an area to be grasped by a user.

17. An apron system configured for hygienic installation of a wound dressing, the apron system comprising:
an apron member including
a front wall and a rear wall defining an inner compartment therebetween;
the rear wall including an aperture providing access to the inner compartment, wherein a border of the aperture is configured to be attached to a patient body; and
at least one glove member integrally attached with the front wall and configured to allow a hand of a user to manipulate items within the inner compartment.

18. The apron system of claim 17, wherein at least a portion of the front wall is substantially transparent and configured to allow a user to view into the inner compartment.

19. The apron system of claim 17, further comprising a removable sealing cover over the aperture and at least one medical supply item.

20. The apron system of claim 17, wherein the inner compartment further comprises a compartment portion configured to receive materials in a manner isolating the materials.
